(19) United States
(12) Patent Application Publication
(10) Pub. No.: US 2020/0305761 A1
(43) Pub. Date: Oct. 1, 2020

(54) PORTABLE SYSTEM FOR MAINSTREAM CAPNOGRAPHY THAT IS CAPABLE OF HANDS-FREE OPERATION

(71) Applicant: Capnography Solutions, LLC, Raleigh, SC (US)

(72) Inventors: Jeffrey Allen Wittkopp, Lakewood, WA (US); Robert Michael Meadows, Honolulu, HI (US)

(21) Appl. No.: 16/830,217

(22) Filed: Mar. 25, 2020

Related U.S. Application Data
(60) Provisional application No. 62/823,599, filed on Mar. 25, 2019.

(51) Int. Cl. A61B 5/097; A61B 5/083; A61B 5/00; A61M 39/10

(52) U.S. Cl. CPC A61B 5/097; A61B 5/0836; A61B 5/6803; A61M 2039/1094; A61M 39/10; A61B 2560/0431; A61B 5/7445

(57) ABSTRACT

A light-weight, portable mask connects to a portable capnography device for monitoring carbon dioxide in expiratory gas. The mask includes an enclosure that fits over the mouth and nose of a patient, and a single outlet to which an expiration lumen is attached. The expiration lumen is configured to attach to the portable capnography device. The expiration lumen and the capnography device are in a position that is parallel to the patient's face when the mask is in use. The portable capnography device is smaller than the mask. The mask may also include a one-way inhalation inlet vent that allows atmospheric air to enter the enclosure when the patient inhales. In this manner, an apparatus including the mask and the capnography device is used to monitor the breathing of a non-intubated patient, and is not configured to supply oxygen to the patient.

### CROSS-REFERENCE TO RELATED APPLICATIONS

This application claims the benefit of U.S. Provisional Application No. 62/823,599, filed on Mar. 25, 2019, entitled “PORTABLE SYSTEM FOR MAINSTREAM CAPNOGRAPHY THAT IS CAPABLE OF HANDS-FREE OPERATION.” The entire content of that application is incorporated herein by reference.

### BACKGROUND

### Field of the Art

This disclosure relates to systems and methods for monitoring the concentration or partial pressure of carbon dioxide in expiratory air output by humans.

### Discussion of the State of the Art

Currently available systems and methods for measuring carbon dioxide levels in humans are inadequate or undesirable for a variety of reasons. Generally, currently available systems can be subdivided into two primary systems, capnography devices that measure carbon dioxide in gas or air respirated by a patient, and pulse oximetry devices that measure carbon dioxide levels in blood stream. Capnography devices can be further categorized as sidestream devices (which use a diverting sampling system to transport a portion of a patient's respired gases from the sampling site, through a sampling tube, to a sensor), and mainstream devices (which use non-diverting measurement system). However, none of these systems may be reliably deployed in a variety of different field conditions because of significant limitations associated with each system.

Sidestream capnography devices, for example, introduce measurement errors due to the very nature of these devices. Generally, the measurements obtained by sidestream capnography devices tend to be inaccurate because a sample typically has to travel from a sampling site to a sensor. Due to the distance between the sampling site (the patient) and the sensor, there is a delay in measuring and recording the expiratory CO₂. In addition, the distance may cause water removal, or the measurement may be inaccurate because the temperature and humidity conditions at the sampling site may be different from the temperature and humidity at the sensor site. Moreover, the sample gas may become mixed as it is drawn through a sensor cell, and cause dynamic distortion to the waveforms. There is a possibility of sampling tube obstruction with sidestream capnography. A pressure drop along the sampling tube affects the CO2 measurements, causing inaccuracies. Although currently available technologies have mitigated the impact of some of these limitations, currently available sidestream devices still suffer from these errors. Moreover, even if these limitations are overcome, sidestream devices nonetheless are still much too heavy and large for deployment in mobile and quickly changing field environments. For example, in a pre-hospital emergency setting, such as a battlefield, where space and resources are limited, a sidestream capnography device is too cumbersome and requires too many steps to set up and obtain a measurement when time is of the essence.

Mainstream capnography devices suffer from similar limitations, and, moreover require a patient to be intubated and ventilated. With mainstream devices, the sensor consisting of the sample cell and infrared bench is placed at the airway. This location results in a better graphical representation of the time varying CO₂ value (capnogram) that reflects in real-time the partial pressure of carbon dioxide within the airway. However, mainstream capnography devices cannot be deployed in a field environment because the devices often require heated cuvette windows and, more importantly, require the patient to be intubated and ventilated. Mainstream capnography devices are difficult to use when a patient is in a prone position. Moreover, these devices are extremely susceptible to user error because any improper connection to other breathing circuit elements can cause artifacts in the capnogram that may be obtained from these devices. In a pre-hospital emergency setting, intubation may be difficult or impossible and if the patient is not intubated, a mainstream capnography device is not an option.

Finally, traditional pulse oximetry devices are generally unreliable and often produce inaccurate results. Moreover, these devices are extremely sensitive to movement and vibration, and, as a result, are not suitable for deployment in fast moving fields such as battlefields and emergency rescue.

Indeed, none of the systems and method that are currently available are suitable for field deployment either because these devices are unsuitable (i.e. either too heavy, too large, too difficult to transport, or too prone to failure in a rapidly evolving field environment). Moreover, currently available mainstream capnography devices require one or more trained personnel to intubate a patient before any capnograms are obtained. Presently the solutions that are available in the marketplace are either difficult to deploy in the field, or require dedicated and trained personnel with specific expertise to administer.

A medical provider in the field may wish to continuously monitor a patient that is able to breathe on their own in order to determine whether the patient needs breathing assistance. Due to limited time and resources, a medical provider in the field would prefer to intubate a patient or use a bag valve mask only when necessary. As such, there is a need for a portable, hands-free apparatus for sampling expiratory gas of a patient that is able to breathe on their own. Such a monitoring device would allow a medical provider to determine when, or if, the patient requires breathing assistance.

### SUMMARY

The present invention is for an apparatus that overcomes the challenges described above with a device that is small and lightweight, provides an accurate measurement of concentration or partial pressure of carbon dioxide in expiratory air output by a patient, can be used in a hands-free manner, and can be deployed in a field environment, including a combat zones, etc.

The present invention accomplishes these objectives by providing an enclosure, an expiratory lumen, a measurement lumen, capnography measurement device, and a retention mechanism. The enclosure fits over the mouth and nose of a user. The expiratory lumen represents an airway exit, and the measurement lumen permits expiration air to travel to the capnography device. In one embodiment of the invention, the measurement lumen and the capnography device are substantially parallel to the patient's body. This configuration enables the inventive apparatus to be used in a hands-free way (by providing a low-profile design). The capnography measurement device is attached to the measurement lumen; it measures the concentration or partial pressure of carbon dioxide in expiratory air output by the patient. The retention mechanism secures the inventive apparatus to the patient's head, thereby ensuring that the inventive apparatus does not move around in quickly changing field environments where the patient may be exposed to different changing environments as well as complicated transport scenarios.

The apparatus of the present invention is suitable for use in the field as a light-weight, portable monitoring device. In one embodiment, the apparatus is for monitoring only and is not configured to supply air to the patient. This embodiment includes a face mask that can be attached to a portable capnography device, but cannot be coupled to an oxygen source.

### BRIEF DESCRIPTION OF THE DRAWING FIGURES

The accompanying drawings illustrate several embodiments and, together with the description, serve to explain the principles of the invention according to the embodiments. It will be appreciated by one skilled in the art that the particular arrangements illustrated in the drawings are merely exemplary and are not to be considered as limiting of the scope of the invention or the claims herein in any way.

FIG. 1 is a front view of an apparatus of the present invention in accordance with an embodiment of the invention showing the mask with the capnography device detached.

FIG. 2 is a side view of an apparatus of the present invention in accordance with an embodiment of the invention showing the mask with the capnography device detached.

FIG. 3 is a front view of an apparatus of the present invention in accordance with an embodiment of the invention showing the capnography device attached to the mask.

FIG. 4 is a side view of an apparatus of the present invention in accordance with an embodiment of the invention showing the capnography device attached to the mask.

FIGS. 5A-5C are, respectively, side, front, and perspective views of an apparatus of the present invention in accordance with an embodiment of the invention.

FIG. 6 is a flow chart depicting a method of using the apparatus of the present invention.

### DETAILED DESCRIPTION

The apparatus of the present invention is for measuring concentration or partial pressure of carbon dioxide in expiratory air output by a patient. The inventive apparatus is designed to ensure that the device can be used and deployed in field environments where it may be difficult to stow and use traditional devices. However, the present invention is not limited to field deployment scenarios. It may be used in any setting, including traditional hospital or healthcare settings.

Generally, one or more different embodiments may be described in the present application. Further, for one or more of the embodiments described herein, numerous alternative arrangements may be described; it should be appreciated that these are presented for illustrative purposes only and are not limiting of the embodiments contained herein or the claims presented herein in any way. One or more of the arrangements may be widely applicable to numerous embodiments, as may be readily apparent from the disclosure. In general, arrangements are described in sufficient detail to enable those skilled in the art to practice one or more of the embodiments, and it should be appreciated that other arrangements may be utilized and that structural, logical, software, electrical and other changes may be made without departing from the scope of the invention.

Particular features of one or more of the embodiments described herein may be described with reference to one or more particular embodiments or figures that form a part of the present disclosure, and in which are shown, by way of illustration, specific arrangements of one or more of the aspects. It should be appreciated, however, that such features are not limited to usage in the one or more particular embodiments or figures with reference to which they are described. The present disclosure is neither a literal description of all arrangements of one or more of the embodiments nor a listing of features of one or more of the embodiments that must be present in all arrangements.

Headings of sections provided in this patent application and the title of this patent application are for convenience only and are not to be taken as limiting the disclosure in any way.

Apparatuses and elements thereof that are connected to each other need not be in continuous connection with each other, unless expressly specified otherwise. In addition, devices and parts that are connected with each other may be connected directly or indirectly through one or more connection means or intermediaries.

A description of an aspect with several components in connection with each other does not imply that all such components are required. To the contrary, a variety of optional components may be described to illustrate a wide variety of possible embodiments and in order to more fully illustrate one or more embodiments. Similarly, although process steps, method steps, or the like may be described in a sequential order, such processes and methods may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the embodiments, and does not imply that the illustrated process is preferred. Also, steps are generally described once per aspect, but this does not mean they must occur once, or that they may only occur once each time a process, or method is carried out or executed. Some steps may be omitted in some embodiments or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other embodiments need not include the device itself.

Techniques and mechanisms described or referenced herein will sometimes be described in singular form for clarity. However, it should be appreciated that particular embodiments may include multiple iterations of a technique or multiple instantiations of a mechanism unless noted otherwise. Alternate implementations are included within the scope of various embodiments in which, for example, functions may be executed out of order from that shown or discussed, including substantially concurrently or in reverse order, depending on the functionality involved, as would be understood by those having ordinary skill in the art

### System Overview

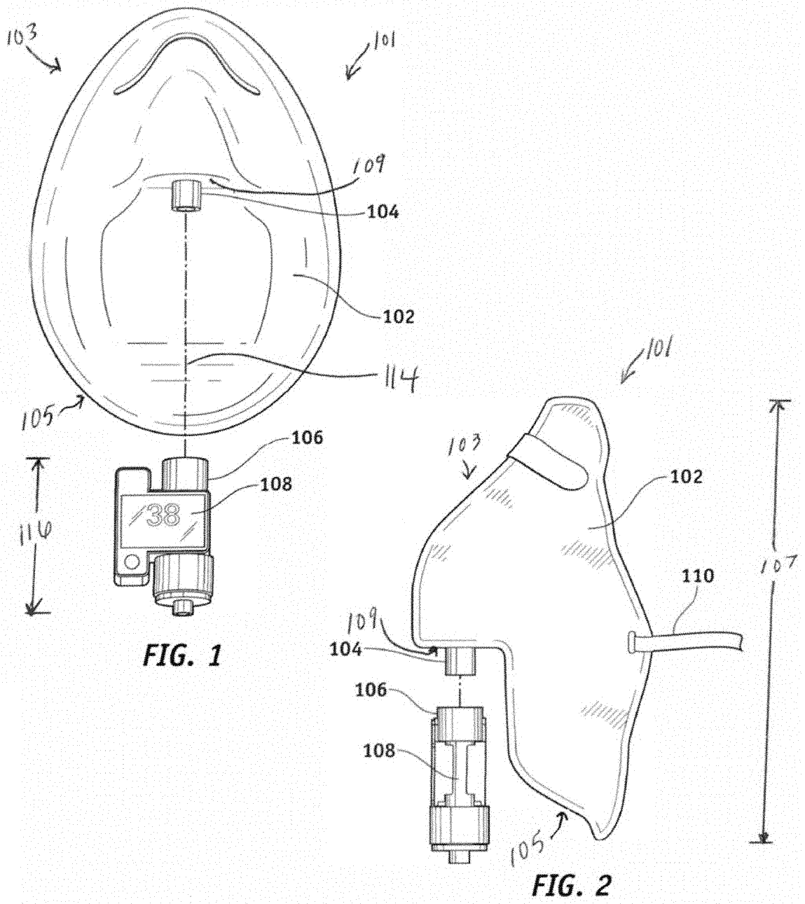

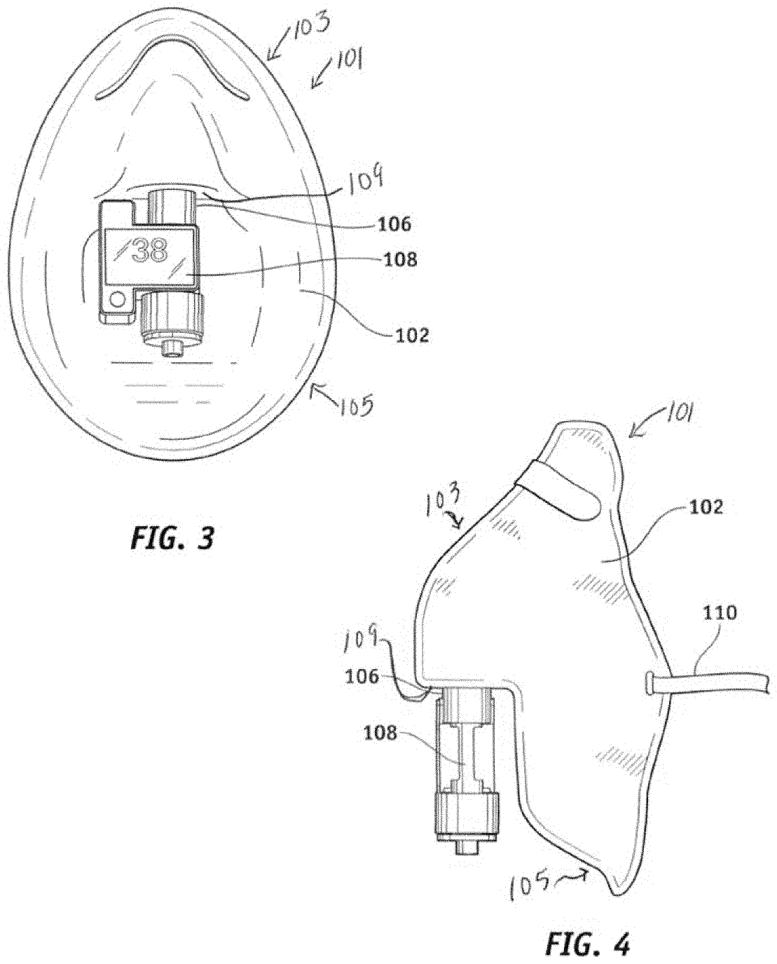

FIGS. 1-4 illustrates the inventive apparatus in accordance with an embodiment of the invention. It is comprised of a mask 101 that includes an enclosure 102, an expiration lumen 104, and a retention device 110. The apparatus further includes a measurement lumen 106 and a capnography measurement device 108 that can be detached from the mask 101. The description provided herein is an exemplary and preferred embodiment of the invention. However, the present invention is not intended to be limited to this particular embodiment. Alternatives, in accordance to the description provided herein, including those that would be readily apparent to a person of ordinary skill in the art are also encompassed in the present disclosure.

### Enclosure

The enclosure 102, in accordance with one embodiment of the invention, fits over the mouth and nose of a patient and prevents air expirated by the patient from interacting with atmospheric air until end tidal CO₂ in the expirated air is measured by the capnography device 108. In one embodiment of the invention, the enclosure 102 creates a seal around the patient's mouth and nose that prevents expirated air from freely exchanging with atmospheric air. In one embodiment of the invention, the enclosure 102 may provide a one-way valve to permit a user to inhale atmospheric air.

The enclosure 102 includes an upper portion 103 designed to fit over the bridge of the nose, and a lower portion 105 designed to contact the patient's chin when the mask 101 is positioned over the nose and mouth. The enclosure has a length 107 that extends from the upper-most edge to the lower-most edge of the enclosure 102. The enclosure 102 also includes a lateral surface 109 that is substantially perpendicular to the patient's face when the mask 101 is in use.

### Expiration and Measurement Lumens

The expiration lumen 104 carries expirated air from within the enclosure 102 to the measurement lumen 106. In one embodiment of the invention, the expiration lumen 104 attaches to the enclosure 102 at or near a point that is downstream from the patient's nostrils. The expiration lumen 104 is attached to the only outlet in the enclosure 102 and has only a single airway or pathway therein. That is, the lumen does not include a divider, sub-chamber, or junction that allows the expiration lumen 104 to be coupled to another device besides the capnography device 108. This ensures that a sufficient amount of expiratory air is delivered to the capnography device 108. As much as possible, air within the enclosure 102 can only exit through the expiration lumen 104. However, since the enclosure 102 does not form an air-tight seal with the patient's face, some air may escape around the edges of the enclosure 102.

The expiration lumen 104 is attached directly to the outlet opening in the enclosure 102. In particular, the expiration lumen 104 has a proximal end that is directly connected to the outlet opening in the enclosure 102, and an open distal end that points towards the lower portion 105 of the enclosure 102 and is sized and shaped to connect to the measurement lumen 106. The expiration lumen 104 is preferably positioned on the lateral surface 109 of the enclosure 102 so that the longitudinal axis 114 of the expiration lumen 104 is substantially parallel with the patient's face when the mask 101 is in use.

The expiration lumen 104 is preferably short so that the capnography device 108 can be attached as close as possible to the patient's face. In one embodiment, the expiration lumen 104 has a length that is less than 1 inch and an outer diameter that is less than 1 inch. The outer diameter of the expiration lumen 104 is slightly smaller than the inner diameter of the measurement lumen 106 to thereby form an air-tight, interference fit connection between the expiration lumen 104 and the measurement lumen 106 with the expiration lumen 104 fitting snugly within the measurement lumen 106. Alternatively, the inner diameter of the expiration lumen 104 may be slightly larger than the outer diameter of the measurement lumen 106 to form the air-tight, interference fit connection between the lumens, with the measurement lumen 106 fitting snugly within the expiration lumen 104.

The measurement lumen 106 extends from the expiration lumen 104 to the capnography measurement device 108. Essentially, the measurement lumen 106 carries expirated air from the expiration lumen 104 to the capnography measurement device 108 such that an accurate reading of the end tidal CO₂ may be made by the measurement device 108. In one embodiment, the measurement lumen 106 is very short, which ensures that air that is measured by the measurement device 108 is substantially the same (in terms of humidity and content) as the expirated air, which further improves the accuracy of the measured end tidal CO₂

### Capnography Measurement Device

The capnography measurement device 108 measures concentration or partial pressure of CO₂ in the air expirated by the patient. More specifically, the capnography measurement device 108 measures the CO₂ in the air expelled through the measurement lumen 106.

A variety of different capnography measurement devices 108 may be used without departing from the scope of the invention to the extent that the devices 108 comply with the disclosure herein. For example, in one embodiment, the capnography device 108 emits infrared light through an adapter window to a photodetector that is located on an opposite side of an airway adapter, wherein the light measured by the photodetector is used to compute end tidal CO₂.

In one embodiment of the invention, the capnography measurement device 108 is substantially parallel to the user or the patient's body. This parallel configuration allows the capnography device 108 and the mask 101 to have a low profile so as to stay out of the way of the medical professional, who may be performing other tasks in close quarters while the mask is attached to the patient.

In one embodiment, the capnography measurement device 108 is strategically placed to balance with the enclosure 102 such that the capnography device 108 does not cause the enclosure 102 to fall off of the patient's face. The orientation and size of the measurement lumen 104 and the capnography device 108 relative to each other and to the enclosure 102 are preferably selected to ensure that the apparatus remains affixed to the patient's face even if the patient is being moved, transported, or jostled around. This is another feature that make the apparatus especially useful in combat and emergency field services environments.

In one embodiment of the invention, the capnography measurement device 108 includes a display, which may be visible to medical professionals around the patient. The capnography device 108 is preferably connected to the mask 101 with the display facing outward, away from the patient's face, so that the medical provider can easily see the information on the display by looking at the patient's face.

The capnography device 108 is small, light-weight, and portable. The capnography device 108 is smaller than the mask 101. In particular, the length 116 of the capnography device 108 is about 40%-60% of the length of the enclosure 102. The capnography device 108 may have a length 116 that is less than half of the length 107 of the enclosure 102. When the capnography device 108 is attached to the mask 101, the distal end of the capnography device 108 preferably does not extend past the lower portion 105 of the enclosure 102. In this manner, the mask 101 with the capnography device 108 attached thereto is a small, portable, unobtrusive apparatus suitable for use in pre-hospital emergency situations, such as a combat zone or the like.

The distal end of the capnography device 108 may be configured to vent aspirated air to the atmosphere. Additionally or alternatively, the capnography device 108 may be configured to block another device or lumen from being attached thereto. For example, the distal end of the capnography device may include a cap, a stopper, a seal, a one-way exit valve, or the like, so that another device cannot be coupled to the distal end of the capnography device 108.

### Retention Mechanism

The retention mechanism 110 secures the apparatus and the enclosure 102 around a patient's mouth and nose. The retention mechanism 110 enables hands-free operation such that a medical professional does not need to hold the apparatus over the patient's face to measure his or her expirated CO₂. This feature enables the medical professionals to perform other care related tasks and/or attend to other matters. More importantly, the retention mechanism 110 ensures that the inventive apparatus is suitable for use in field environments such as combat zones, in transport vehicles, etc.

In one embodiment, the retention mechanism 110 may be a strap that hooks around the back of the patient's head and connects to two opposite ends of the enclosure 102. Other retention mechanisms, which may be readily understood by persons of ordinary skill in the art, may be used without departing from the scope of the invention.

### Efficacy

Studies conducted by the applicants with a version of the inventive apparatus have shown that the results obtained with the inventive concept are correlated to sidestream capnography. This correlation was validated utilizing several statistical analysis models showing a high confidence value of accuracy. Also, this study was found to be safe with no subjects observed becoming hypoxic or having difficulty breathing. The ability to assess capnography on a patient that is not intubated or ventilated would be extremely beneficial in identifying life-saving interventions needed. This ability is not currently available as previously discussed.

Accordingly, the system disclosed herein can be used in the field and can be used to measure end tidal CO₂ in a patient that is not intubated. In contrast to a conventional sidestream capnography system, the current system allows a medical provider to monitor a patient's end tidal CO₂ by looking at the patient rather than having to look away at a monitor. The current system is also more portable, more light-weight, less obtrusive, and easier to use than a conventional sidestream capnography system. The current system is also advantageous over conventional mainstream capnography systems because the current system does not require that the patient be intubated.

The system disclosed herein is used to monitor a patient's breathing and to determine whether the patient needs breathing assistance. Since the system is only used for monitoring, the enclosure 102 includes only one air outlet, and a monitoring device 108 is coupled to that outlet. The system does not include an inlet that is coupled to, or configured to be coupled to, an oxygen source. However, the system may include an inlet that is configured to allow atmospheric air into the enclosure when the patient inhales.

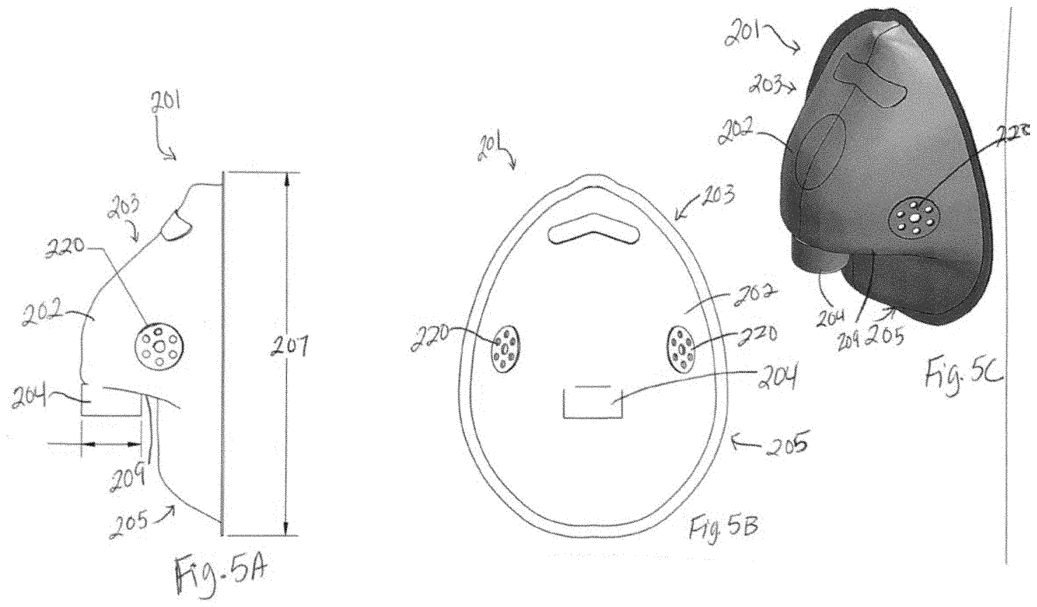

In particular, in another embodiment, shown in FIGS. 5A-5C, the apparatus includes one way inhalation vents 220 disposed on the sides of the enclosure 202 slightly above the expiration lumen 204. The inhalation vents 220 may include a diaphragm or the like that allows air to flow into the enclosure through the inlet vents 220, and prevents air from flowing out of the enclosure 202 through the inlet vents 220. Although two inlet vents 220 are depicted, it should be well understood that the mask 201 may alternatively include only one inlet vent or may include more than two inlet vents without departing from the spirit and scope of the invention.

Similar to the mask 101 described above, the mask 201 depicted in FIGS. 5A-5C includes an enclosure 202 having an upper portion 203 and a lower portion 205. The mask 201 also includes an expiration lumen 204 disposed between the upper portion 203 and the lower portion 205. The enclosure 202 includes a lateral surface 209 that is perpendicular to the patient's face when the mask 201 is attached to the patient's face. The expiration lumen 204 is attached to the lateral surface 209 so that the longitudinal axis of the expiration lumen 204 and the capnography device attached thereto are in a position that is parallel to the patient's face. In this manner, the apparatus maintains a low profile so as to be as unobtrusive as possible to a medical provider in the field.

The length 207 of the enclosure 202 is much greater than the length of a capnography device to be attached to the expiration lumen 204. Preferably, when the capnography device is attached to the expiration lumen 204, the distal end of the capnography device does not extend past the lower portion 205 of the enclosure 202. Alternatively, the capnography device extends a very small distance past the lower portion of the enclosure 202.

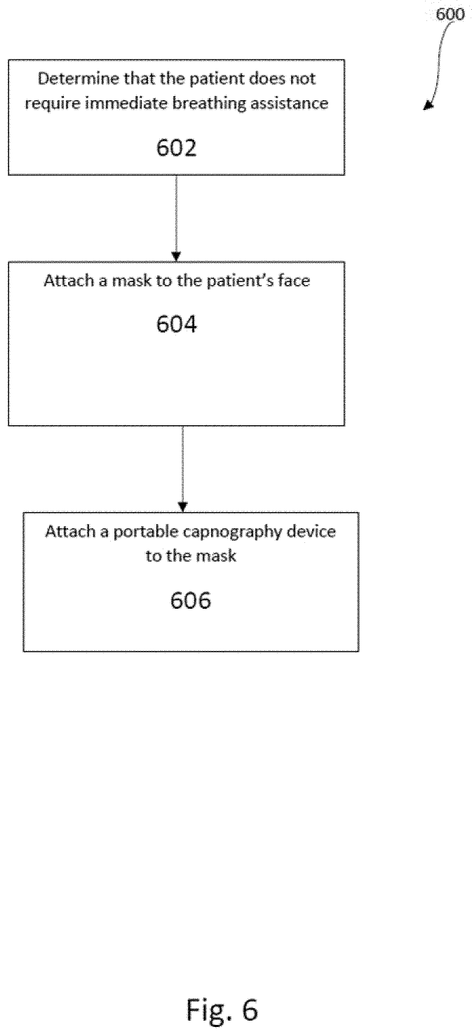

The apparatuses disclosed herein are used in a method for monitoring the breathing of a non-intubated patient. The method 600 is depicted in FIG. 6. First, in step 602, the medical provider determines that the patient does not require immediate breathing assistance. As discussed elsewhere herein, in a combat zone or other such pre-hospital emergency situation, the medical provider would prefer not to intubate the patient or provide breathing assistance with a bag valve mask. These breathing assistance measures take up precious time and resources in an emergency situation. Nonetheless, the medical provider may still want to monitor a patient's breathing in case the patient may subsequently require breathing assistance. As such, in step 604, a mask, such as the masks 101, 201 disclosed herein, is attached to the patient's face to cover the nose and mouth of the patient. Finally, in step 606, a portable capnography device, such as the capnography device 108 disclosed herein, is attached to the mask for monitoring the concentration or partial pressure of CO2 in the patient's expiratory air. One of ordinary skill in the art would understand that steps 604 and 606 may occur simultaneously, or in reverse order. That is, the capnography device may be coupled to the mask before the mask is placed over the patient's nose and mouth.

### Additional Considerations

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and Bis false (or not present), A is false (or not present) and Bis true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and a process for creating an interactive message through the disclosed principles herein. Thus, while particular embodiments and applications have been illustrated and described, it is to be understood that the disclosed embodiments are not limited to the precise construction and components disclosed herein. Various apparent modifications, changes and variations may be made in the arrangement, operation and details of the method and apparatus disclosed herein without departing from the spirit and scope defined in the appended claims.

What is claimed is:

1. A light-weight and portable apparatus for hands-free measurement of concentration or partial pressure of carbon dioxide in expiratory air output by a non-intubated patient, the apparatus comprising:
an enclosure that fits over the mouth and nose of the patient;
an expiratory lumen for directing expirated air to exit the enclosure;
a measurement lumen that is connected to the expiratory lumen, the measurement lumen being substantially parallel to the patient's face when the enclosure is positioned over the nose and mouth;
a capnography device connected to the measurement lumen for measuring concentration or partial pressure of carbon dioxide in expiratory air output by a patient, the capnography device being substantially parallel to the patient's face when the enclosure is positioned over the nose and mouth; and
a retention device for securing the enclosure to the patient's face, the retention device ensuring that expirated air does not leak out of the enclosure except through the expiratory lumen, and the retention device ensuring that the apparatus may be used in a hands-free way.

2. The apparatus of claim 1, wherein the capnography device comprises a display for displaying the measured concentration or partial pressure, and wherein the capnography device is positioned with the display pointing outward and away from the patient's face so that a medical provider can easily read the display when looking at the patient's face.

3. The apparatus of claim 1, wherein the enclosure comprises a single outlet and the expiratory lumen is attached to the single outlet.

4. The apparatus of claim 3, wherein the enclosure comprises at least one inlet having a one-way valve for allowing atmospheric air to enter the enclosure through the inlet and preventing air from exiting the enclosure through the inlet.

5. The apparatus of claim 4, wherein the one-way valve is configured to allow atmospheric air to enter the enclosure through the inlet when the patient inhales.

6. The apparatus of claim 1, wherein the capnography device is smaller than the enclosure.

7. The apparatus of claim 1, wherein a length of the capnography device is 40%-60% of a length of the enclosure

8. The apparatus of claim 1, wherein the capnography device has a proximal end that is attached to the measurement lumen, and a distal end that does not extend past a bottom edge of the enclosure.

9. The apparatus of claim 1, wherein a distal end of the capnography device vents aspirated air to the atmosphere.

10. The apparatus of claim 1, wherein a distal end of the capnography device is configured to block attachment of another lumen or device thereto.

11. A mask configured to connect to a portable capnography device for performing a mainstream end tidal CO₂ measurement on a patient, wherein the portable capnography device is smaller than the mask, and wherein the mask comprises:
an enclosure that fits over the mouth and nose of the patient, wherein the enclosure has an upper portion that fits over the nose of the patient, a lower portion that is configured to be in contact with a chin of the patient, a lateral surface disposed between the upper portion and the lower portion, and a single outlet disposed in the lateral surface, wherein the lateral surface is substantially perpendicular to the patient's face when the enclosure is positioned over the patient's nose and mouth; and
an expiratory lumen directly coupled to the single outlet of the enclosure and positioned so that a longitudinal axis of the lumen is substantially parallel to the patient's face when the enclosure is secured to the patient's face, wherein the lumen is positioned to direct expiratory air from inside the enclosure to the capnography device, wherein the expiratory lumen is sized and shaped to form a substantially air-tight connection with the portable capnography device.

12. The mask of claim 11, wherein an outer diameter of the expiratory lumen is less than 1 inch.

13. The mask of claim 11, wherein the expiratory lumen has a length that is less than 1 inch.

14. The mask of claim 11, wherein the expiratory lumen comprises a proximal end that is directly attached to the single outlet in the enclosure and a distal end that points towards the lower portion of the enclosure.

15. The mask of claim 11, wherein the expiratory lumen comprises only a single pathway to ensure that a sufficient amount of expiratory air is delivered to the capnography device.

16. The mask of claim 11, wherein the enclosure comprises at least one inlet having a one-way gasket that allows atmospheric air to flow into the enclosure through the inlet and prevents air from flowing out of the enclosure through the inlet.

17. The mask of claim 11, wherein the mask is configured for monitoring the patient's breathing, but is not configured for coupling to an oxygen source.

18. The mask of claim 16, wherein the at least one inlet is a vent that cannot be coupled to an oxygen source.

19. A method for monitoring a patient's breathing, the method comprising:
determining that the patient does not require breathing assistance;
attaching a mask to cover the patient's nose and mouth, wherein the mask includes a single outlet and at least one inlet having a one-way valve for allowing atmospheric air to enter the mask through the inlet and preventing air from exiting the mask through the inlet; and
attaching a portable capnography device to the single outlet of the mask, wherein the portable capnography device is smaller than the mask.

20. The method of claim 19, wherein the single outlet is disposed in a lateral surface of the mask, wherein the lateral surface is substantially perpendicular to the patient's face when the mask is attached to the patient's nose and mouth, wherein an expiration lumen is coupled to the single outlet, and wherein attaching the portable capnography device to the single outlet comprises attaching the portable capnography device directly to the expiration lumen such that the portable capnography device is disposed in a position that is substantially parallel to the patient's face.
